(19) United States
(12) Patent Application Publication
(10) Pub. No.: US 2019/0275098 A1
(43) Pub. Date: Sep. 12, 2019

(54) NOVEL FORMULATIONS AND USES THEREFOR

(71) Applicant: SOLANA MAR SKIN SCIENCE, LLC, Del Mar, CA (US)

(72) Inventor: Maria BONILLA, Del Mar, CA (US)

(21) Appl. No.: 16/461,786

(22) PCT Filed: Nov. 16, 2017
(86) PCT No.: PCT/US2017/062075

§ 371 Date: May 16, 2019

Related U.S. Application Data
(60) Provisional application No. 62/423,076, filed on Nov. 16, 2016.

(51) Int. Cl. A61K 36/88; A61P 17/08; A61K 47/10; A61K 47/14; A61P 17/12

(52) U.S. Cl. CPC A61K 36/88; A61P 17/08; A61K 2236/15; A61K 47/14; A61P 17/12; A61K 47/10

(57) ABSTRACT

In accordance with the present disclosure, there are provided methods for obtaining an active agent useful for the topical treatment of benign epidermal condition(s) in a subject in need thereof. In accordance with certain aspects of the present disclosure, there are provided active agents obtained by the methods described herein. In certain aspects, there are provided formulations for the topical treatment of benign epidermal condition(s) in a subject in need thereof. In certain aspects, there are provided methods for the preparation of formulations for the topical treatment of benign epidermal condition(s) in a subject in need thereof. In certain aspects, there are provided methods for the topical treatment of benign epidermal condition(s) in a subject in need thereof.

### FIELD OF THE DISCLOSURE

The present disclosure relates to methods for obtaining active agents useful for the topical treatment of benign epidermal condition(s) in a subject in need thereof. In certain aspects, the present disclosure relates to active agents obtained by the methods described herein. In certain aspects, the present disclosure relates to formulations for the topical treatment of benign epidermal condition(s) in a subject in need thereof. In certain aspects, the present disclosure relates to methods for the preparation of formulations for the topical treatment of benign epidermal condition(s) in a subject in need thereof. In certain aspects, the present disclosure relates to methods for the topical treatment of benign epidermal condition(s) in a subject in need thereof.

### BACKGROUND OF THE DISCLOSURE

The information provided herein and references cited are provided solely to assist the understanding of the reader, and does not constitute an admission that any of the references or information is prior art to the present disclosure.

Seborrheic Keratoses are one of the most common benign epidermal growths in adults. It is estimated that by age sixty-five, 88% of adults will have at least one lesion. They can begin to appear as early as age 25. In general, people tend to accumulate these growths as they get older. Some individuals can acquire 50 or more lesions in their lifetime. Genetics, ultraviolet light exposure, and friction all play a role in determining how many a person will accumulate. They often have a warty appearance, so it is no wonder that they are commonly referred to as barnacles on the ship of life. In fact, during the Victorian era, they were commonly known as senile warts. This makes them cosmetically unacceptable for many people. Also, it is not uncommon for individual lesions to become pruritic, irritated, and/or traumatized. In addition, they can appear virtually overnight. It is for these reasons that many individuals with seborrheic keratoses seek out dermatologists for their removal. Currently, treatment options are limited to cryotherapy, microdermabrasion, electrodessication, laser, radiofrequency ablation, surgical removal, and topical treatments. Procedural treatment options can be expensive and include a risk of bleeding, infection, post-inflammatory pigment alteration, and scarring. While there are many topical options, none are very effective.

### SUMMARY OF THE DISCLOSURE

In accordance with the present disclosure, there are provided methods for obtaining active agents useful for the topical treatment of benign epidermal condition(s) in a subject in need thereof. There are also provided active agents obtained by the methods described herein. In certain aspects, there are provided formulations for the topical treatment of benign epidermal condition(s) in a subject in need thereof. In certain aspects, there are provided methods for the preparation of formulations for the topical treatment of benign epidermal condition(s) in a subject in need thereof. In certain aspects, there are provided methods for the topical treatment of benign epidermal condition(s) in a subject in need thereof.

In one aspect, the disclosure provides a method for obtaining an active agent useful for the topical treatment of a benign epidermal condition in a subject in need thereof. The method comprises: grinding banana peel into a paste; adding 0.5-10 volumes (e.g., 0.5-9, 0.5-8, 0.5-7, 0.5-6, 0.5-5, 0.5-4, 0.5-3, 0.5-2, or 0.5-1 volumes) of a suitable diluent to said paste, thereby producing a substantially homogeneous suspension; separating macroparticulate matter from said substantially homogeneous suspension; and adding an antioxidant to the resulting substantially macroparticulate-free suspension.

In some embodiments, the active agent is banana peel extract (Musa sp.). The banana peel extract may be obtained from a ripe banana. The banana peel extract may be obtained from a green banana. The banana peel extract may be obtained from a mixture of green and ripe bananas. In certain embodiments, the ratio of green banana peel to ripe banana peel falls in the range of about 0.1-10:1 (e.g., 0.2-10:1, 0.3-10:1,. 0.4-10:1, 0.5-10:1, 0.6-10:1, 0.7-10:1, 0.8-10:1, 0.9-10:1, or 1-10:1).

The active agent (e.g., banana peel extract (Musa sp.)) obtained by methods described herein may be used to treat a benign epidermal condition selected from seborrheic keratoses, verrucae vulgaris, warty dyskeratomas, inverted follicular keratoses, benign lichenoid keratoses, porokeratoses, hyperkeratotic dermatoses, epidermal nevi, purpura, acne, skin brightening, dermatitis, pruritus, xerosis, seborrheic dermatitis, Fitzpatrick skin type II lesion and/or growth, Fitzpatrick skin type III lesion and/or growth, Fitzpatrick skin type IV lesion and/or growth, dermatosis papulosa nigrans, or acneiform papules.

In some embodiments, the suitable diluent used in methods of obtaining the active agent (e.g., banana peel extract (Musa sp.)) is water. In some embodiments, the suitable solvent is an ester of an unsaturated, long chain fatty acid. In particular embodiments, the ester of an unsaturated, long chain fatty acid is heptyl undecenylate.

The disclosure also features the active agent obtained by the methods described in the previous aspect.

In another aspect, the disclosure provides a formulation for the topical treatment of a benign epidermal condition in a subject in need thereof, comprising: the active agent of the previous aspect, and a pharmaceutically acceptable carrier therefor.

In another aspect, the disclosure provides a formulation for the topical treatment of a benign epidermal condition in a subject in need thereof, comprising a clay ingredient and a pharmaceutically acceptable carrier therefor. The formulation containing the clay ingredient may be used to occlude the formulation containing the active agent after the formulation containing the active agent is applied onto the skin of the subject. The clay ingredient may be selected from the group consisting of calcium bentonite, bentonite clay, Australian black clay, blue montmorillonite clay, Dead sea clay, French green clay, Fuller's Earth clay, kaolin white clay (China clay), kaolin red clay, kaolin pink clay, kaolin yellow clay, illite clay, moor mud clay (heilmoor clay), rhassoul clay, rose clay, and sea clay.

In some embodiments, formulations described herein may further comprise organic solvents, amino acids, vitamins, minerals, essential oils, alpha and beta hydroxy acids, carboxylic or keto acids, enzymes, co-enzymes, botanical actives, and/or organic oxides and reductants.

In some embodiments, formulations described herein may further comprise one or more vehicle selected from the group consisting of caprylic/capric triglycerides, heptyl undecylenate, anhydrous alcohol, oils, esters and solvents.

In some embodiments, the amount of vehicle in a formulation may be sufficient for the formulation to form a solution, a gel, a serum, an emulsion, an ointment, a spray, or a paste.

In some embodiments, formulations described herein may further comprise one or more of an antioxidant (e.g., tocopherol), a stabilizer, salicylic acid, benzoic acid, almond oil, bentonite, kukui oil, urea, tea tree oil, rose hip oil, Dead sea mud, and/or Dead sea salt.

In another aspect, the disclosure provides a method for the preparation of a formulation for the topical treatment of a benign epidermal condition in a subject in need thereof, comprising combining a sufficient quantity of the active agent described herein (e.g., banana peel extract (Musa sp.)), and optionally a pharmaceutically acceptable carrier therefor, with a sufficient quantity of a suitable vehicle under conditions suitable to form a solution, a gel, a serum, an emulsion, an ointment, a spray, or a paste.

In another aspect, the disclosure provides a method for the topical treatment of a benign epidermal condition in a subject in need thereof, comprising: optionally pre-treating the skin with a retinoid, urea, glycolic acid, salicylic acid, dilute acetic acid, hydrogen peroxide, witch hazel, and/or apple cider vinegar, and thereafter, applying an effective amount of the active agent described herein (e.g., banana peel extract (Musa sp.)).

In another aspect, the disclosure provides a method for the topical treatment of a benign epidermal condition in a subject in need thereof, comprising: optionally pre-treating the skin with a retinoid, urea, glycolic acid, salicylic acid, dilute acetic acid, hydrogen peroxide, witch hazel, and/or apple cider vinegar, and thereafter, applying an effective amount of the active agent described herein (e.g., banana peel extract (Musa sp.)), and occluding the active agent with a clay ingredient.

In some embodiments, an effective amount of the active agent (e.g., banana peel extract (Musa sp.)) falls in the range of 0.001 g to 10 g (e.g., 0.001 g to 8 g, 0.001 g to 6 g, 0.001 g to 4 g, 0.001 g to 2 g, 0.001 g to 1 g, 0.01 g to 10 g, 0.1 g to 10 g, 1 g to 10 g, 2 g to 10 g, 3 g to 10 g, 4 g to 10 g, 5 g to 10 g, 6 g to 10 g, 7 g to 10 g, 8 g to 10 g, or 9 g to 10 g) applied once or twice daily.

In another aspect, the disclosure provides a method for the topical treatment of a benign epidermal condition in a subject in need thereof, comprising: optionally pre-treating the skin with a retinoid, urea, glycolic acid, salicylic acid, dilute acetic acid, hydrogen peroxide, witch hazel, and/or apple cider vinegar, and thereafter, applying an effective amount of the formulation containing an active agent (e.g., banana peel extract (Musa sp.)).

In another aspect, the disclosure provides a method for the topical treatment of a benign epidermal condition in a subject in need thereof, comprising: optionally pre-treating the skin with a retinoid, urea, glycolic acid, salicylic acid, dilute acetic acid, hydrogen peroxide, witch hazel, and/or apple cider vinegar, and thereafter, applying an effective amount of the formulation containing an active agent (e.g., banana peel extract (Musa sp.)), and occluding the formulation containing the active agent with a clay formulation comprising a clay ingredient.

In some embodiments, the clay ingredient is selected from the group consisting of calcium bentonite, bentonite clay, Australian black clay, blue montmorillonite clay, Dead sea clay, French green clay, Fuller's Earth clay, kaolin white clay (China clay), kaolin red clay, kaolin pink clay, kaolin yellow clay, illite clay, moor mud clay (heilmoor clay), rhassoul clay, rose clay, and sea clay.

The benign epidermal condition that may be treated by methods described herein include, e.g., seborrheic keratoses, verrucae vulgaris, warty dyskeratomas, inverted follicular keratoses, benign lichenoid keratoses, porokeratoses, hyperkeratotic dermatoses, epidermal nevi, purpura, acne, skin brightening, dermatitis, pruritus, xerosis, seborrheic dermatitis, Fitzpatrick skin type II lesion and/or growth, Fitzpatrick skin type III lesion and/or growth, Fitzpatrick skin type IV lesion and/or growth, dermatosis papulosa nigrans, or acneiform papules.

### DETAILED DESCRIPTION OF THE DISCLOSURE

The disclosure provides formulations and methods for treating benign epidermal condition(s) in a subject in need thereof. In accordance with certain aspects of the present disclosure, there are provided active agents (e.g., banana peel extract (Musa sp.)). A clay formulation may also be used to occlude the active agent or the formulation containing thereof after the active agent or the formulation containing thereof is applied.

### Active Agents

In accordance with the present disclosure, there are provided methods for obtaining active agents useful for the topical treatment of benign epidermal condition(s) in a subject in need thereof. The disclosure provides methods comprising:

grinding banana peel into a paste;

adding about 0.5-10 volumes (e.g., 0.5-9, 0.5-8, 0.5-7, 0.5-6, 0.5-5, 0.5-4, 0.5-3, 0.5-2, or 0.5-1 volumes) of a suitable diluent to said paste, thereby producing a substantially homogeneous suspension;

separating macroparticulate matter from said substantially homogeneous suspension; and

adding an antioxidant to the resulting substantially macroparticulate-free suspension.

In some embodiments, the active agent is banana peel extract (Musa sp.). In some embodiments, the banana peel extract is obtained from ripe banana(s). In some embodiments, the banana peel extract is obtained from green banana(s).

In some embodiments, the banana peel extract is obtained from a mixture of green and ripe bananas. When mixtures of green and ripe bananas are employed, the ratio thereof can vary substantially. Typically, the ratio of green banana peel to ripe banana peel falls in the range of about 0.1-10:1. In some aspects, the ratio of green banana peel to ripe banana peel falls in the range of about 0.2-10:1; in some aspects, the ratio of green banana peel to ripe banana peel falls in the range of about 0.3-10:1; in some aspects, the ratio of green banana peel to ripe banana peel falls in the range of about 0.4-10:1; in some aspects, the ratio of green banana peel to ripe banana peel falls in the range of about 0.5-10:1; in some aspects, the ratio of green banana peel to ripe banana peel falls in the range of about 0.6-10:1; in some aspects, the ratio of green banana peel to ripe banana peel falls in the range of about 0.7-10:1; in some aspects, the ratio of green banana peel to ripe banana peel falls in the range of about 0.8-10:1; in some aspects, the ratio of green banana peel to ripe banana peel falls in the range of about 0.9-10:1; in some aspects, the ratio of green banana peel to ripe banana peel falls in the range of about 1-10:1.

Suitable diluents contemplated for use herein include water, ethanol, 2-propanol, sweet almond oil, apricot kernel oil, avocado oil, babassu oil, olive oil, safflower seed oil, sesame seed oil, soybean oil, sunflower oil, jojoba oil, jojoba esters, isopropyl myristate, isopropyl palmitate, C₁₂₋₁₅ alkyl benzoate, caprylyl/capryl triglyceride, cetyl ethylhexanoate, cetearyl ethylhexanoate, dimethyl isosorbide, ethoxydiglycol, ethylhexyl palmitate, polyethylene glycol, propylene glycol, butylene glycol, pentylene glycol, propanediol, and the like, as well as mixtures of any two or more thereof.

In some aspects, in the range of about 0.5-10 volumes of a suitable diluent are employed relative to the amount of paste; in some aspects, in the range of about 1-10 volumes of a suitable diluent are employed relative to the amount of paste; in some aspects, in the range of about 1-8 volumes of a suitable diluent are employed relative to the amount of paste; in some aspects, in the range of about 1.5-6 volumes of a suitable diluent are employed relative to the amount of paste. The term “volume” as used herein refers to a measurement of the amount or quantity of a component in a suspension, solution, or formulation. A volume of a component may be measured by any unit that measures the three-dimensional space occupied by the component, e.g., a cup, a tablespoon, a teaspoon, liter, milliliter, cubic meter, or cubic centimeter. The volume of a component may be measured relative to the volume of another component in the suspension, solution, or formulation. When measuring the volume of a component relative to the volume of another component, volume(s) of a component may be interpreted as part(s) of the component. For example, 0.5-10 volumes of a suitable diluent may be 0.5-10 parts of a suitable diluent per 1 part of the banana peel paste. Each part may be measured by any unit that measures the three-dimensional space occupied by the component, e.g., a cup, a tablespoon, a teaspoon, liter, milliliter, cubic meter, or cubic centimeter.

Substantially homogeneous suspensions achieved by the grinding process contemplated herein typically comprise a suspension of particulate material, wherein the majority of the particulate material has a particle size in the range of about 0.5 up to about 500 microns. In some embodiments, the majority of the particulate material has a particle size in the range of about 0.5 up to about 400 microns. In some embodiments, the majority of the particulate material has a particle size in the range of about 0.5 up to about 300 microns. In some embodiments, the majority of the particulate material has a particle size in the range of about 0.5 up to about 200 microns. In some embodiments, the majority of the particulate material has a particle size in the range of about 0.5 up to about 100 microns. In some embodiments, the majority of the particulate material has a particle size in the range of about 10 up to about 500 microns. In some embodiments, the majority of the particulate material has a particle size in the range of about 10 up to about 400 microns. In some embodiments, the majority of the particulate material has a particle size in the range of about 10 up to about 300 microns. In some embodiments, the majority of the particulate material has a particle size in the range of about 10 up to about 200 microns. In some embodiments, the majority of the particulate material has a particle size in the range of about 10 up to about 100 microns.

Particulate matter substantially larger than the range set forth above (i.e., macroparticulate matter) is separated from said substantially homogeneous suspension by suitable means, e.g., by using a sufficiently small mesh size filter (e.g., a Grade 60 Mesh, which comprises 32×28 threads per inch), thereby producing a substantially macroparticulate-free suspension.

Antioxidants contemplated for addition to the resulting substantially macroparticulate-free suspension include tocopherol, L-ascorbic acid, ferulic acid, caffeine, resveratrol, retinol, green tea polyphenols, coffee berry, licorice root, coenzyme Q10, essential oils, and the like.

In accordance with another embodiment of the present disclosure, there are provided active agents obtained by the methods described herein. Depending on the ratio of green/ripe peels used in the preparation of the formulation, one or more of the following may also be present in the resulting formulation, i.e., leucocyanidin, dopamine, tryptophan, lutein, lignin, ascorbic acid, tocopherol, carotenes, phenolic compounds like gallocatechin, and the like.

### Formulations for the Topical Treatment of Benign Epidermal Conditions

In accordance with yet another embodiment of the present disclosure, there are provided formulations for the topical treatment of a benign epidermal condition in a subject in need thereof. The disclosure provides formulations comprising the active agent described herein, and a pharmaceutically acceptable carrier therefor.

In accordance with yet another embodiment of the present disclosure, there are provided clay formulations for the topical treatment of a benign epidermal condition in a subject in need thereof. A clay formulation may be used after the formulation comprising the active agent and the pharmaceutically acceptable carrier is already applied. In some embodiments, a clay formulation may be applied to occlude the formulation comprising the active agent and the pharmaceutically acceptable carrier as described in the examples. The clay formulation may provide structural support to help the formulation comprising the active agent maintain in place on the subject's skin. A clay formulation may include a clay ingredient and also a pharmaceutically acceptable carrier.

Examples of clay ingredients that may be used in this aspect of the disclosure include, but are not limited to, calcium bentonite, bentonite clay, Australian black clay, blue montmorillonite clay, Dead sea clay, French green clay, Fuller's Earth clay, kaolin white clay (China clay), kaolin red clay, kaolin pink clay, kaolin yellow clay, illite clay, moor mud clay (heilmoor clay), rhassoul clay, rose clay, and sea clay.

Pharmaceutically acceptable carriers contemplated for use herein include solutions, tinctures, sera, emulsions, gels, lotions, creams, ointments, foams, powder, tapes, sprays, pastes, and the like, as well as mixtures of any two or more thereof. A pharmaceutically acceptable carrier can contain physiologically acceptable agents that act, for example, to stabilize or to increase the absorption of a compound such as a compound of the disclosure. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The pharmaceutical composition (preparation) also can be a liposome or other polymer matrix, which can have incorporated therein, for example, a compound of the disclosure. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

In some embodiments, the formulations (e.g., the formulation comprising an active agent or the clay formulation) may further comprise organic solvents, amino acids, vitamins, minerals, essential oils, alpha and beta hydroxy acids, carboxylic or keto acids, enzymes, co-enzymes, botanical actives, organic oxides and reductants, and the like, as well as mixtures of any two or more thereof.

Organic solvents contemplated for use herein include isopropyl myristate, isopropyl palmitate, C₁₂₋₁₅ alkyl benzoate, dimethyl isosorbide, ethoxydiglycol, ethylhexyl palmitate, polyethylene glycol, butylene glycol, pentylene glycol, propanediol, propylene glycol, acetic acid, methanol, 2-propanol, glycerin, and the like, as well as mixtures of any two or more thereof.

Amino acids contemplated for use herein include L-arginine, L-glycine, L-histidine, L-isoleucine, L-leucine, L-lysine, L-proline, L-tyrosine, L-valine, and the like, as well as mixtures of any two or more thereof.

Vitamins contemplated for use herein include ascorbic acid, tocopherol, niacinamide, thiamine, riboflavin, retinyl acetate, pyridoxine, and the like, as well as mixtures of any two or more thereof.

Minerals contemplated for use herein include copper, magnesium, zinc oxide, titanium dioxide, iron oxides, kaolinite, and the like, as well as mixtures of any two or more thereof.

Essential oils contemplated for use herein include argan oil, sweet almond, coconut, tea tree, lemon balm, lemon grass, rose, eucalyptus, pomegranate seed, grapeseed, apricot kernel, carrot seed, lavender, geranium, sunflower seed, avocado, pistachio vera seed, rose hip seed, and the like, as well as mixtures of any two or more thereof.

Alpha and beta hydroxy acids contemplated for use herein include glycolic, citric. tartaric, lactic, mandelic, salicylic, and the like, as well as mixtures of any two or more thereof.

Carboxylic or keto acids contemplated for use herein include acetic acid, benzoic acid, pyruvic acid, phytic acid, and the like, as well as mixtures of any two or more thereof.

Enzymes contemplated for use herein include fruit enzymes (e. g., papaya), and the like, as well as mixtures of any two or more thereof.

Co-enzymes contemplated for use herein include Q10, and the like, as well as mixtures of any two or more thereof.

Botanical actives contemplated for use herein include arbutin, aloesin, flavonoids, hesperidin, licorice, niacinamide, coffee berry, chamomile, soy, witch hazel, yeast derivatives, polyphenols, and the like, as well as mixtures of any two or more thereof.

Organic oxides and reductants contemplated for use herein include hydrogen peroxide, zinc, and the like, as well as mixtures of any two or more thereof.

Additional components contemplated for use herein include wetting or emulsifying agents, pH buffering agents, and the like.

In some embodiments, the formulations (e.g., the formulation comprising an active agent or the clay formulation) may further comprise one or more vehicle selected from the group consisting of caprylic/capric triglycerides, heptyl undecylenate, anhydrous alcohol, oils, esters and solvents.

In some embodiments, the formulations (e.g., the formulation comprising an active agent or the clay formulation) may contain a sufficient quantity of said vehicle so as to form a solution, a tincture, a gel, a serum, an emulsion, a lotion, a cream, an ointment, a foam, a powder, a tape a spray, a paste, or the like.

In some embodiments, the formulations (e.g., the formulation comprising an active agent or the clay formulation) may further comprise one or more of an antioxidant (e.g., tocopherol), a stabilizer, salicylic acid, benzoic acid, almond oil, bentonite, kukui oil, urea, tea tree oil, rose hip oil, Dead sea mud, Dead sea salt, and the like, as well as mixtures of any two or more thereof.

In some embodiments, the formulations (e.g., the formulation comprising an active agent or the clay formulation) may further comprise one or more additional ingredients which include, but are not limited to, rosemary, sage, thyme, lemon balm, aloe vera, burdock root, cat's claw, chaga nushroom, coconut, dandelion, nettle leaf, raw honey, red clover, rose hips, wild blueberries, brazil nuts, calendula, chamomile, chrysanthemum, cleaver, cloves, coconut water, cumin, dong quai, echinacea, eye bright, fenugreek, gotu kola, guava, honeysuckle, lavender, linden flowers, lomatium, mullein, marshmallow root, mustard oil, nasturium, oatstraw, persimmons, pumpkin, raspberry leaf, roses, red root, sage, star anise, sweet violet, tumeric, and watercress.

### Methods for Preparing the Formulations

In accordance with still another embodiment of the present disclosure, there are provided methods for the preparation of formulations for the topical treatment of benign epidermal condition(s) in a subject in need thereof. The methods comprise combining a sufficient quantity of the active agent described herein, and optionally a pharmaceutically acceptable carrier therefor, with a sufficient quantity of a suitable vehicle under conditions suitable to form a solution, a tincture, a serum, an emulsion, a gel, a lotion, a cream, an ointment, a foam, a powder, a tape, a spray, a paste, or the like.

### Methods for the Topical Treatment of Benign Epidermal Conditions

In accordance with yet another embodiment of the present disclosure, there are provided methods for the topical treatment of a benign epidermal condition, e.g., seborrheic keratosis, a wart, or Fitzpatrick skin type II, III, or IV lesion and/or growth, in a subject in need thereof. The methods comprise:

optionally pre-treating the skin with an alcohol, a retinoid (e.g. tretinoin), urea, an alpha-hydroxy acid, a beta-hydroxy acid, dilute acetic acid, hydroquinone, hydrogen peroxide, witch hazel, and/or apple cider vinegar, and thereafter

applying an effective amount of an active agent (e.g., banana peel extract (Musa sp.)) as described herein.

In accordance with yet another embodiment of the present disclosure, there are provided methods for the topical treatment of a benign epidermal condition, e.g., seborrheic keratosis, a wart, or Fitzpatrick skin type II, III, or IV lesion and/or growth, in a subject in need thereof. The methods comprise:

optionally pre-treating the skin with an alcohol, a retinoid (e.g. tretinoin), urea, an alpha-hydroxy acid, a beta-hydroxy acid, dilute acetic acid, hydroquinone, hydrogen peroxide, witch hazel, and/or apple cider vinegar,

applying an effective amount of an active agent (e.g., banana peel extract (Musa sp.)) as described herein, and

occluding the active agent with a clay ingredient.

Examples of clay ingredients that may be used in this aspect of the disclosure include, but are not limited to, calcium bentonite, bentonite clay, Australian black clay, blue montmorillonite clay, Dead sea clay, French green clay, Fuller's Earth clay, kaolin white clay (China clay), kaolin red clay, kaolin pink clay, kaolin yellow clay, illite clay, moor mud clay (heilmoor clay), rhassoul clay, rose clay, and sea clay.

Benign epidermal conditions contemplated for treatment herein include, but are not limited to, seborrheic keratoses, verrucae vulgaris, warty dyskeratomas, inverted follicular keratoses, benign lichenoid keratoses, porokeratoses, hyperkeratotic dermatoses, purpura, acne, skin brightening, dermatitis, pruritus, xerosis, seborrheic dermatitis, Fitzpatrick skin type II lesion and/or growth, Fitzpatrick skin type III lesion and/or growth, Fitzpatrick skin type IV lesion and/or growth, dermatosis papulosa nigrans, acneiform papules, and the like. Benign epidermal conditions contemplated for treatment herein also include, e.g., skin wounds, abrasions, lesions, cuts, sores, papules, and/or lacerations.

Exemplary subjects contemplated for treatment herein include subjects who have been exposed to, or are susceptible to formation of seborrheic keratoses, verrucae vulgaris, warty dyskeratomas, inverted follicular keratoses, benign lichenoid keratoses, porokeratoses, hyperkeratotic dermatoses, epidermal nevi, purpura, acne, skin brightening, dermatitis, pruritus, xerosis, seborrheic dermatitis, Fitzpatrick skin type II lesion and/or growth, Fitzpatrick skin type III lesion and/or growth, Fitzpatrick skin type IV lesion and/or growth, dermatosis papulosa nigrans, acneiform papules, and the like.

A variety of treatment protocols can be employed herein, for example, an effective amount of active agent contemplated for use herein will typically fall in the range of about 0.001 up to about 10 g applied once or twice daily.

In accordance with yet another embodiment of the present disclosure, there are provided methods for the topical treatment of a benign epidermal condition, e.g., seborrheic keratosis, a wart, or Fitzpatrick skin type II, III, or IV lesion and/or growth, in a subject in need thereof. The methods comprise:

optionally pre-treating the skin with an alcohol, a retinoid (e.g. tretinoin), urea, an alpha-hydroxy acid, a beta-hydroxy acid, dilute acetic acid, hydroquinone, hydrogen peroxide, witch hazel, and/or apple cider vinegar, and thereafter

applying an effective amount of a formulation comprising an active agent (e.g., banana peel extract (Musa sp.)) as described herein.

In accordance with yet another embodiment of the present disclosure, there are provided methods for the topical treatment of a benign epidermal condition, e.g., seborrheic keratosis, a wart, or Fitzpatrick skin type II, III, or IV lesion and/or growth, in a subject in need thereof. The methods comprise:

optionally pre-treating the skin with an alcohol, a retinoid (e.g. tretinoin), urea, an alpha-hydroxy acid, a beta-hydroxy acid, dilute acetic acid, hydroquinone, hydrogen peroxide, witch hazel, and/or apple cider vinegar, and thereafter

applying an effective amount of a formulation comprising an active agent (e.g., banana peel extract (Musa sp.)) as described herein, and

occluding the formulation comprising the active agent with a clay formulation.

A clay formulation may include a clay ingredient. Examples of clay ingredients that may be used in this aspect of the disclosure include, but are not limited to, calcium bentonite, bentonite clay, Australian black clay, blue montmorillonite clay, Dead sea clay, French green clay, Fuller's Earth clay, kaolin white clay (China clay), kaolin red clay, kaolin pink clay, kaolin yellow clay, illite clay, moor mud clay (heilmoor clay), rhassoul clay, rose clay, and sea clay.

In methods of treating benign epidermal condition(s) described herein, the amount and/or frequency of use of the active agent (e.g., banana peel extract (Musa sp.)) or the formulation containing thereof, and optionally the clay formulation used to occlude the active agent, may be depend on factors including the type of the epidermal condition and/or the severity of the epidermal condition. The amount and/or frequency of use may be decided by the affected subject or a physician.

In any of the methods described herein for the topical treatment of benign epidermal condition(s) in a subject, the active agent (e.g., banana peel extract (Musa sp.)) or the formulation containing thereof, and optionally the clay formulation used to occlude the active agent, may be left on the skin of the subject for as long as needed. The active agent (e.g., banana peel extract (Musa sp.)) or the formulation containing thereof, and optionally the clay formulation may also be reapplied during the treatment period as needed. In some embodiments, the active agent (e.g., banana peel extract (Musa sp.)) or the formulation containing thereof, and optionally the clay formulation may also be reapplied once daily, once every two days, once every four days, once every six days, once a week, once every two weeks, or once a month.

In some embodiments, the active agent (e.g., banana peel extract (Musa sp.)) or the formulation containing thereof, and optionally the clay formulation may be left on the subject's skin for at least half a day, at least one day, at least two days, at least three days, at least four days, at least five days, at least six days, at least seven days, at least eight days, at least nine days, at least ten days, at least fifteen days, at least twenty days, at least thirty days, at least forty days, at least fifty days, or at least sixty days. In some embodiments, the active agent (e.g., banana peel extract (Musa sp.)) or the formulation containing thereof, and optionally the clay formulation may be left on the subject's skin for between one to twenty days, between one to fifteen days, between one to ten days, between one to eight days, between one to six days, between one to four days, between one to two days, between two to twenty days, between four to twenty days, between six to twenty days, between eight to twenty days, between ten to twenty days, between fifteen to twenty days, between two to fifteen days, between four to ten days, or between six to eight days.

Kits

The disclosure also provides kits that include (1) the active agent (e.g., banana peel extract (Musa sp.)) or the formulation containing thereof, and (2) instructions for applying (1) to a subject with an epidermal condition. In some embodiments, the kits may also include a clay ingredient (or the formulation containing thereof) and instructions for applying the clay ingredient (or the formulation containing thereof). The active agent (e.g., banana peel extract (Musa sp.)) (or the formulation containing thereof) and the clay ingredient (or the formulation containing thereof) may be packaged in the same kit or in separate kits.

In certain embodiments, the kits may include (1) a formulation containing the active agent (e.g., banana peel extract (Musa sp.)), (2) a formulation containing a clay ingredient, and (3) instructions for applying (1) and (2) to a subject with an epidermal condition.

The kit can include optional components that aid in the application of the formulations, such as swabs, bandages, gauzes, adhesive strips, sponges, and/or patches. The kit components may be assembled in cartons, blister packs, bottles, tubes, and the like. The kit may be manufactured as a single use for a unit dose or multiple uses for multiple doses.

The following examples are provided to further illustrate aspects of the disclosure. These examples are non-limiting and should not be construed as limiting any aspect of the disclosure.

### EXAMPLES

### Example 1

A seborrheic keratosis lesion on the back of a 73 year old male with a Fitzpatrick skin type III lesion was treated with a solution according to the present disclosure (comprising 50% ripe banana peel extract and 50% anhydrous alcohol) on a nightly basis for a week. After 1 week the treated lesion began to break apart and fall off the skin.

### Example 2

A wart on the 2^{nd} toe of a 9 year old male with a Fitzpatrick skin type III growth was treated with a solution according to the present disclosure (comprising 50% green banana peel extract and 50% anhydrous alcohol) and occluded with Dead sea clay overnight for 3 nights. On the fourth morning, the lesion had developed a crust that fell-off 2 days later.

### Example 3

Seborrheic keratoses on the bilateral cheeks of a 59 year old female with a Fitzpatrick skin type IV lesion was treated with a solution according to the present disclosure (comprising 50% ripe banana peel extract and 50% caprylic/capric triglycerides) and lesions were subsequently occluded with bentonite clay overnight for 7 nights. On the eighth morning, over 50% of the lesions had developed crusting that fell-off during that week.

### Example 4

Regional small seborrheic keratoses on the bilateral neck of a 70 year old female with a Fitzpatrick skin type III lesion were treated with a solution according to the present disclosure (comprising 50% ripe banana peel extract and 50% caprylic/capric triglycerides). The solution was applied daily at bedtime and subsequently covered with clay. This was washed off in the mornings. The lesions slowly began to break apart and ultimately fell-off over a period of 2 weeks.

### Example 5

Stucco variant of seborrheic keratoses on the bilateral dorsal feet of a 45 year old male with a Fitzpatrick skin type III lesion were treated with a solution according to the present disclosure (comprising 50% ripe banana peel extract and 50% caprylic/capric triglycerides) and lesions were subsequently occluded with clay overnight for 13 nights. By the end of the second week, over 70 percent of lesions had either developed a crust and/or fallen-off.

### Example 6

A pigmented seborrheic keratosis on the right temple of a 52 year old male with a Fitzpatrick skin type III lesion was treated with a solution according to the present disclosure (comprising 50% ripe banana peel extract and 50% Heptyl Undecylenate) twice daily for 16 nights. At that point, the lesion had fallen-off.

### Example 7

A flat wart on the foot of a 44 year old male with skin type III was treated with a solution according to the present disclosure (comprising 50% green banana peel extract and 50% anhydrous alcohol) and occluded with clay overnight for 4 nights. The lesion fell-off on the fifth morning.

### Example 8

Purpuric patches on the bilateral forearms of a 59 year old female with a Fitzpatrick skin type II lesion were treated with a solution according to the present disclosure (comprising 50% ripe banana peel extract and 50% caprylic/capric triglycerides). The solution was applied daily at bedtime and subsequently covered with Dead sea clay. This was washed off in the mornings. Purpura began to fade over a period of a week.

### Example 9

Abrasions on the bilateral knees of a 24 year old male with a Fitzpatrick skin type II lesion were treated with a solution according to the present disclosure (comprising 50% ripe banana peel extract and 50% caprylic/capric triglycerides) and lesions were subsequently occluded with green clay overnight for 5 nights. By the 6th day, the crust fallen-off.

### Example 10

Multiple dermatosis papulosa nigrans (DPN) on the bilateral malar cheeks of a 56 year old female with a Fitzpatrick skin type III-IV lesion were treated with a solution according to the present disclosure (comprising 50% green/ripe 1:1 banana peel extract and 50% heptyl undecylenate) daily for 20 nights. After 3 weeks the DPNs had fallen-off.

### Example 11

An acneiform papule on the chin of a 24 year old female with skin type III was treated with a solution according to the present disclosure (comprising 50% green banana peel extract and 50% anhydrous alcohol) and occluded with Fuller's earth clay overnight for 2 nights. The lesion had resolved on the 3rd morning.

The disclosure illustratively described herein may be practiced in the absence of any element or elements, limitation or limitations which is not specifically disclosed herein. Thus, for example, the terms “comprising”, “including,” containing”, etc. shall be read expansively and without limitation. Additionally, the terms and expressions which have been employed are used as terms of description and not of limitation, and there is no intention in the use of such terms and expressions of excluding any equivalents of the features shown and described or portions thereof, but it is recognized that various modifications are possible within the scope of the disclosure claimed. Accordingly, it should be understood that although the present disclosure has been specifically disclosed by preferred embodiments and optional features, modification and variation of the concepts herein disclosed may be resorted to by those skilled in the art, and that such modifications and variations are considered to be within the scope of this disclosure as defined by the appended claims.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other documents.

The disclosure has been described broadly and generically herein. Each of the narrower species and subgeneric groupings falling within the generic disclosure also form part of the disclosure. This includes the generic description of the disclosure with a proviso or negative limitation removing any subject matter from the genus, regardless of whether or not the excised material is specifically recited herein.

In addition, where features or aspects of the disclosure are described in terms of Markush groups, those skilled in the art will recognize that the disclosure is also thereby described in terms of any individual member or subgroup of members of the Markush group.

Other embodiments are set forth within the following claims.

Although the disclosure is illustrated and described herein with reference to specific embodiments, the disclosure is not intended to be limited to the details shown. Rather, various modifications may be made in the details within the scope and range of equivalents of the claims without departing from the disclosure.

1-24. (canceled)

25. A method for obtaining an active agent, said method comprising:
a. grinding peel from a banana (Musa sp.) into a paste;
b. adding 0.5-10 volumes of a diluent to said paste, thereby producing a substantially homogeneous suspension;
c. removing macroparticulate matter from said substantially homogeneous suspension, thereby producing a substantially microparticulate-free suspension; and
d. adding an antioxidant to the substantially macroparticulate-free suspension;
wherein the active agent is a banana (Musa sp.) peel extract that is useful for topical treatment of a benign epidermal condition in a subject in need thereof.

26. The method of claim 25, wherein the peel is obtained from a ripe banana.

27. The method of claim 25, wherein the peel is obtained from a green banana.

28. The method of claim 25, wherein the peel is obtained from a mixture of green and ripe bananas.

29. The method of claim 28, wherein the ratio of green banana peel to ripe banana peel is about 0.1-10:1.

30. The method of claim 25, wherein the diluent is selected from the group consisting of: water, ethanol, 2-propanol, sweet almond oil, apricot kernel oil, avocado oil, babassu oil, olive oil, safflower seed oil, sesame seed oil, soybean oil, sunflower oil, jojoba oil, jojoba esters, isopropyl myristate, isopropyl palmitate, C₁₂₋₁₅ alkyl benzoate, caprylyl/capryl triglyceride, cetyl ethylhexanoate, cetearyl ethylhexanoate, dimethyl isosorbide, ethoxydiglycol, ethylhexyl palmitate, polyethylene glycol, propylene glycol, butylene glycol, pentylene glycol, and propanediol

31. The method of claim 30, wherein the diluent is water.

32. An active agent obtained by the method of claim 25.

33. A formulation for the topical treatment of a benign epidermal condition in a subject in need thereof, comprising the active agent of claim 32.

34. The formulation of claim 33, wherein the formulation comprises a pharmaceutically acceptable carrier.

35. The formulation of claim 33, wherein the formulation comprises an organic solvents, an amino acids, a vitamins, a minerals, an essential oils, an alpha hydroxy acid, a beta hydroxy acids, a carboxylic acid, a keto acid, an enzyme, a co-enzymes, a botanical actives, an organic oxides, an organic reductants or a mixture of any two or more thereof.

36. The formulation of claim 33, wherein the formulation comprises one or more vehicle selected from the group consisting of heptyl undecylenate, anhydrous alcohol, oils, esters and solvents.

37. The formulation of claim 36, containing a sufficient quantity of the one or more vehicle to form a solution, a gel, a serum, an emulsion, an ointment, a spray, or a paste.

38. The formulation of claim 33, wherein the formulation comprises one or more antioxidant, stabilizer, salicylic acid, benzoic acid, almond oil, bentonite, kukui oil, urea, tea tree oil, rose hip oil, Dead sea mud, or Dead sea salt.

39. A method for the preparation of a formulation for the topical treatment of a benign epidermal condition in a subject in need thereof, said method comprising combining the active agent of claim 32 with a sufficient quantity of a vehicle to form a solution, a gel, a serum, an emulsion, an ointment, a spray, or a paste.

40. A method for the topical treatment of a benign epidermal condition in a subject in need thereof, said method comprising the step of:
a. applying an effective amount of the active agent of claim 32 to the skin of the subject.

41. The method of claim 40, further comprising pre-treating the skin with a retinoid, urea, glycolic acid, salicylic acid, dilute acetic acid, hydrogen peroxide, witch hazel, and/or apple cider vinegar prior to step a.

42. The method of claim 40, further comprising the step of:
b. occluding the active agent with a clay ingredient following step a.

43. The method of claim 42, wherein the clay ingredient is selected from the group consisting of calcium bentonite, bentonite clay, Australian black clay, blue montmorillonite clay, Dead sea clay, French green clay, Fuller's Earth clay, kaolin white clay (China clay), kaolin red clay, kaolin pink clay, kaolin yellow clay, illite clay, moor mud clay (heilmoor clay), rhassoul clay, rose clay, and sea clay.

44. The method of claim 40, wherein the benign epidermal condition is selected from the group consisting of: seborrheic keratoses, verrucae vulgaris, warty dyskeratomas, inverted follicular keratoses, benign lichenoid keratoses, porokeratoses, hyperkeratotic dermatoses, epidermal nevi, purpura, acne, skin brightening, dermatitis, pruritus, xerosis, seborrheic dermatitis, Fitzpatrick skin type II lesions and/or growths, Fitzpatrick skin type III lesions and/or growths, Fitzpatrick skin type IV lesions and/or growths, dermatosis papulosa nigrans, and acneiform papules.
